(19) United States
(12) Patent Application Publication
(10) Pub. No.: US 2023/0355398 A1
(43) Pub. Date: Nov. 9, 2023

(54) JOINT ARTHROPLASTY IMPLANTS, SYSTEMS, AND METHODS

(71) Applicant: RTG Scientific, LLC, Austin, TX (US)

(72) Inventors: Andrew FAUTH, North Logan, UT (US); Richard Justin HYER, Hyrum, UT (US); Jonathan BITTER, Hyde Park, UT (US)

(21) Appl. No.: 18/143,032

(22) Filed: May 3, 2023

Related U.S. Application Data
(60) Provisional application No. 63/338,263, filed on May 4, 2022.

(51) Int. Cl. A61F 2/30

(52) U.S. Cl. CPC A61F 2/30771; A61F 2002/30863; A61F 2002/30886; A61B 17/72

(57) ABSTRACT

A joint replacement implant may include a shaft having a proximal end, a distal end, and a threaded shaft portion, a helical thread disposed about the shaft defining a length of the threaded shaft portion and including a concave undercut surface angled towards one of the proximal end and the distal end of the shaft, and an attachment feature at the proximal end of the joint replacement implant. The attachment feature may be configured to couple an articular member to the joint replacement implant. The articular member may include at least one artificial articular surface that may be shaped to replace at least a portion of a natural articular surface of a joint of a patient.

### CROSS-REFERENCE TO RELATED APPLICATIONS

This application claims the benefit of U.S. Provisional Patent Application Ser. No. 63/338,263 filed on May 4, 2022, entitled “JOINT ARTHROPLASTY IMPLANTS, SYSTEMS, AND METHODS.” The foregoing document is hereby incorporated by reference in its entirety.

### TECHNICAL FIELD

The present disclosure relates to joint arthroplasty implants, systems, and methods. More specifically, the present disclosure relates to joint arthroplasty implants with improved thread designs for increasing bone fixation and load sharing.

### BACKGROUND

Joint arthroplasty procedures are conducted to restore the function of an unhealthy joint. Typically, these procedures involve replacing the unhealthy natural articular surfaces of the joint with artificial articular surfaces. The new artificial articular surfaces are typically anchored into the adjacent bones to maintain long term stability. However, joint arthroplasty devices implanted within bone can become lose over time due to compression forces, radial outward loading forces, multi-axial forces, and/or off-axis loading scenarios that may be applied to the joint arthroplasty device during the healing process. Joint arthroplasty implants utilizing traditional thread designs, tapered stems, keels, or other methods may not provide sufficient fixation to overcome these forces and off-axis loading scenarios.

Accordingly, joint arthroplasty implants with improved thread designs for increasing bone fixation and load sharing between a bone/implant interface experiencing multi-axial forces and off-loading conditions would be desirable. Such improved thread designs can provide improved fixation, purchase, stability, stiffness, and yield strength, as well as decreased wear and degradation of the bone surrounding the implant.

### SUMMARY

The various joint arthroplasty implants, systems, and methods of the present disclosure have been developed in response to the present state of the art, and in particular, in response to the problems and needs in the art that have not yet been fully solved by currently available joint arthroplasty implants, systems, and methods. In some embodiments, the joint arthroplasty implants, systems, and methods of the present disclosure may provide improved fixation and load sharing characteristics between a bone/implant interface experiencing multi-axial and off-loading conditions.

In some embodiments, a joint replacement implant may include a shaft having a proximal end, a distal end, and a threaded shaft portion, a helical thread disposed about the shaft defining a length of the threaded shaft portion and including a concave undercut surface angled towards one of the proximal end and the distal end of the shaft, and an attachment feature at the proximal end of the joint replacement implant. The attachment feature may be configured to couple an articular member to the joint replacement implant. The articular member may include at least one artificial articular surface and the at least one artificial articular surface may be shaped to replace at least a portion of a natural articular surface of a joint of a patient.

In some embodiments, the attachment feature may include at least one of: a post, a Morse taper feature, a passageway, a dovetail feature, a tray feature, a snap-fit feature, and a tab feature.

In some embodiments, the joint replacement implant may also include an intermediary component couplably disposed intermediate the attachment feature and the articular member.

In some embodiments, the intermediary component may include at least one of: an intermediary neck component and an intermediary tray component.

In some embodiments, the joint replacement implant may be configured to receive at least one of: a pin, a fastener, and a keel. The pin, the fastener, and the keel may be configured to stabilize the joint replacement implant relative to a bone of the patient.

In some embodiments, the shaft of the joint replacement implant may be shaped to be at least partially received within an intramedullary canal of a bone of the patient.

In some embodiments, the shaft may include a tapered shaft portion and the helical thread may include a tapered helical thread disposed about the tapered shaft portion.

In some embodiments, a joint replacement implant system may include a joint replacement implant and an articular member coupled to the proximal end of the joint replacement implant. The joint replacement implant may include a shaft having a proximal end, a distal end, and a threaded shaft portion and a helical thread disposed about the shaft defining a length of the threaded shaft portion. The helical thread may include a concave undercut surface angled towards one of the proximal end and the distal end of the shaft. The articular member may include at least one artificial articular surface, and the at least one artificial articular surface may be shaped to replace at least a portion of a natural articular surface of a joint of a patient.

In some embodiments, the joint replacement implant system may also include an attachment feature at the proximal end of the joint replacement implant configured to couple the articular member to the joint replacement implant.

In some embodiments, the joint replacement implant system may also include an intermediary component disposed intermediate the attachment feature and the articular member and configured to couple therebetween.

In some embodiments, the joint replacement implant system may also include at least one of: a pin, a fastener, and a keel. The pin, the fastener, and the keel may be configured to stabilize the joint replacement implant relative to a bone of the patient.

In some embodiments, the articular member may include a femoral head prosthesis and the artificial articular surface may include a convex semi-spherical artificial articular surface.

In some embodiments, the articular member may include a tibial insert prosthesis and the artificial articular surface may include one or more concave artificial articular surfaces.

In some embodiments, the articular member may include an ankle insert prosthesis and the artificial articular surface may include at least one of a concave artificial articular surface and a convex artificial articular surface.

In some embodiments, a method for replacing at least a portion of a natural articular surface of a joint of a patient may include preparing a bone of the patient to receive a joint replacement implant therein and inserting the joint replacement implant into the bone of the patient. The joint replacement implant may include a shaft having a proximal end, a distal end, and a threaded shaft portion, a helical thread disposed about the shaft defining a length of the threaded shaft portion including a concave undercut surface angled towards one of the proximal end and the distal end of the shaft, and an attachment feature at the proximal end of the joint replacement implant. The attachment feature may be configured to couple an articular member to the joint replacement implant. The articular member may include at least one artificial articular surface and the at least one artificial articular surface may be shaped to replace at least a portion of a natural articular surface of a joint of a patient.

In some embodiments, preparing the bone to receive the joint replacement implant may further include forming a bone tunnel in the bone configured to receive at least a portion of the joint replacement implant therein.

In some embodiments, the method may also include tapping a helical bone thread about the bone tunnel formed in the bone.

In some embodiments, inserting the joint replacement implant into the bone may further include placing the helical thread of the joint replacement implant adjacent the helical bone thread tapped about the bone tunnel, and rotating the joint replacement implant relative to the bone tunnel to insert the helical thread into the helical bone thread and insert the joint replacement implant into the bone tunnel.

In some embodiments, inserting the joint replacement implant into the bone may further include placing the helical thread of the joint replacement implant adjacent the bone tunnel, and rotating the joint replacement implant relative to the bone tunnel to form a helical bone thread about the bone tunnel (via one or more self-tapping features of the joint replacement implant) and to insert the joint replacement implant into the bone tunnel.

In some embodiments, the method may also include coupling the articular member to the attachment feature of the joint replacement implant.

These and other features and advantages of the present disclosure will become more fully apparent from the following description and appended claims or may be learned by the practice of the joint arthroplasty implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1A illustrates a front perspective view of a fastener, according to an embodiment of the present disclosure; FIG. 1B illustrates a rear perspective view of the fastener of FIG. 1A;

FIG. 1C illustrates a side view of the fastener of FIG. 1A; FIG. 1D illustrates a cross-sectional side view of the fastener of FIG. 1A taken along the line A-A shown in FIG. 1C;

FIG. 2 illustrates a partial cross-sectional side view of a fastener comprising crescent-shaped threading;

FIG. 3 illustrates a tapered bone implant, according to an embodiment of the present disclosure;

FIGS. 4A-4D illustrate various hip joint implants, according to embodiments of the present disclosure;

FIGS. 5A and 5B illustrate various views of a hip joint implant, according to another embodiment of the present disclosure;

FIG. 6A and FIG. 6B illustrate various views of a hip joint implant, according to another embodiment of the present disclosure;

FIG. 7 illustrates a hip joint implant, according to another embodiment of the present disclosure;

FIG. 8 illustrates a hip joint implant, according to another embodiment of the present disclosure;

FIGS. 9A-9H illustrate various tibial knee joint implants, according to embodiments of the present disclosure;

FIG. 10 illustrates a femoral knee joint implant, according to an embodiment of the present disclosure; and

FIGS. 11A-11C illustrate various views of an ankle joint implant, according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the implants, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

As used herein, the terms fastener, bone implant, joint arthroplasty implant, etc., can comprise any device (having any structure or shape) implantable within bone that may (or may not) utilize any thread morphology described or contemplated herein, including, but not limited to: bone screws, pedicle screws, headless/headed screws, interference screws, compression screws, lag screws, long screws, half pins/Schanz fasteners, cannulated screws, threaded stems, threaded intramedullary canal bone implants, threaded joint replacement implants (e.g., hip joint implants, knee joint implants, ankle joint implants, hand/foot/finger/toe joint implants, shoulder joint implants, elbow joint implants, spinal joint implants, etc.), bone anchors, soft tissue repair anchors, etc.

It will also be understood that any fastener, bone implant, joint arthroplasty implant, etc., described or contemplated herein may (or may not) include any thread configuration, feature, or morphology described or contemplated herein to achieve optimal fixation within a given bone. Moreover, it will also be understood that any fastener, bone implant, joint arthroplasty implant, etc., described or contemplated herein may be utilized in conjunction with (or within) any system, method, procedure, and/or instrumentation described or contemplated herein.

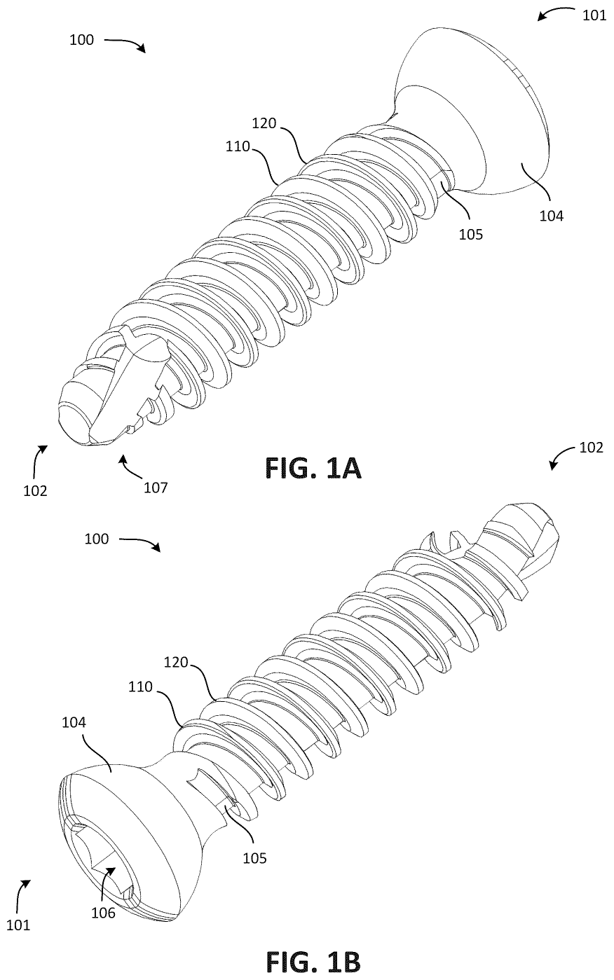

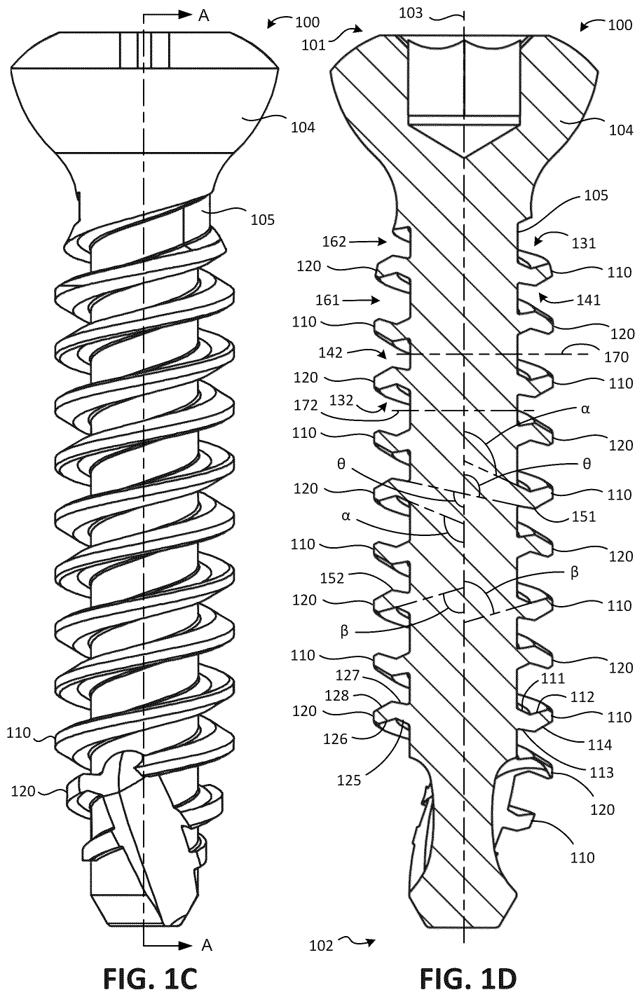

FIGS. 1A-1D illustrate various views of a bone implant, joint arthroplasty implant, or fastener 100, according to an embodiment of the present disclosure. Specifically, FIG. 1A is a front perspective view of the fastener 100, FIG. 1B is a rear perspective view of the fastener 100, FIG. 1C is a side view of the fastener 100, and FIG. 1D is a cross-sectional side view of the fastener 100 taken along the line A-A in FIG. 1C.

In general, the fastener 100 may include a shaft 105 having a proximal end 101, a distal end 102, and a longitudinal axis 103. The fastener 100 may also include a head 104 located at the proximal end 101 of the shaft 105, a torque connection interface 106 formed in/on the head 104 (in either a male/female configuration), and a self-tapping feature 107 formed in the distal end 102 of the shaft 105.

In some embodiments, the fastener 100 may include a first helical thread 110 disposed about the shaft 105, and a second helical thread 120 disposed about the shaft 105 adjacent the first helical thread 110.

In some embodiments, the fastener 100 may include a “dual start” or “dual lead” thread configuration comprising the first helical thread 110 and the second helical thread 120.

In some embodiments, a depth of the first helical thread 110 and/or the second helical thread 120 with respect to the shaft 105 may define a major diameter vs. a minor diameter of the shaft 105 alone.

In some embodiments, a major diameter and/or a minor diameter of the fastener 100 may be constant or substantially constant along the entire length of the fastener, or along a majority of the length of the fastener. In these embodiments, a constant minor diameter may help avoid blowout of narrow/delicate bones (e.g., a pedicle) when inserting a fastener into a bone. In some embodiments, a pilot hole may first be drilled into a narrow/delicate bone and then a fastener having a similar minor diameter in comparison to the diameter of the pilot hole may be chosen to avoid blowout when inserting the fastener into the bone, as will be discussed in more detail below.

In some embodiments, a depth of the first helical thread 110 and/or the second helical thread 120 with respect to the shaft 105 may vary along a length of the shaft 105 to define one or more major diameters of the fastener 100 and/or one or more regions along the fastener 100 may comprise one or more continuously variable major diameters.

In some embodiments, a thickness of the shaft 105 may vary along a length of the shaft 105 to define one or more minor diameters of the fastener 100, and/or one or more regions along the fastener 100 may comprise one or more continuously variable minor diameters.

In some embodiments, a thickness/height/width/length/pitch/angle/shape, etc., of the first helical thread 110 and/or the second helical thread 120 (or any additional helical thread) may vary along a length of the shaft 105. For example, a thickness/height/width/length/pitch/angle/shape, etc., of the first helical thread 110 and/or the second helical thread 120 may be greater towards the tip of the fastener and thinner towards the head of the fastener (or vice versa) in either a discrete or continuously variable fashion, etc.

In some embodiments, the major and/or minor diameters may increase toward a proximal end or head of a fastener in order to increase bone compaction as the fastener is terminally inserted into the bone/tissue.

In some embodiments, a pitch of the first helical thread 110 and/or the second helical thread 120 may vary along a length of the fastener 100.

In some embodiments, the fastener 100 may include a plurality of helical threads disposed about the shaft 105. However, it will also be understood that any of the fasteners disclosed or contemplated herein may include a single helical thread disposed about the shaft of the fastener. Moreover, the fastener 100 may comprise a nested plurality of helical threads having different lengths (not shown). As one non-limiting example, the fastener 100 may include a first helical thread 110 that is longer than a second helical thread 120, such that the fastener 100 comprises dual threading along a first portion of the shaft 105 and single threading along a second portion of the shaft 105.

In some embodiments, the plurality of helical threads may include three helical threads comprising a “triple start” or “triple lead” thread configuration (not shown).

In some embodiments, the plurality of helical threads may include four helical threads comprising a “quadruple start” or “quadruple lead” thread configuration (not shown).

In some embodiments, the plurality of helical threads may include more than four helical threads (not shown).

In some embodiments, the fastener 100 may include first threading with any of the shapes disclosed herein oriented toward one of the proximal end and the distal end of the fastener 100, with the first threading located proximate the distal end of the fastener 100, as well as second threading with any of the shapes disclosed herein oriented toward the other one of the proximal end and the distal end of the fastener 100, with the second threading located proximate the head of the fastener 100 (not shown).

In some embodiments, the fastener 100 may include multiple threading (e.g., dual helical threading, etc.) with any of the shapes disclosed herein located proximate one of the proximal end and the distal end of the fastener 100, as well as single threading with any of the shapes disclosed herein with the second threading located proximate the other of the proximal end and the distal end of the fastener 100.

In some embodiments, the first helical thread 110 may include a plurality of first concave undercut surfaces 131 and a plurality of first convex undercut surfaces 141.

In some embodiments, the second helical thread 120 may include a plurality of second concave undercut surfaces 132 and a plurality of second convex undercut surfaces 142.

In some embodiments, when the fastener 100 is viewed in section along a plane that intersects the longitudinal axis 103 of the shaft 105 (e.g., see FIG. 1D), the plurality of first concave undercut surfaces 131 and the plurality of second convex undercut surfaces 142 may be oriented toward (i.e., point toward) the proximal end 101 of the shaft 105.

In some embodiments, the plurality of first convex undercut surfaces 141 and the plurality of second concave undercut surfaces 132 may be oriented toward (i.e., point toward) the distal end 102 of the shaft 105.

In some embodiments, at least one of the plurality of first concave undercut surfaces 131, the plurality of first convex undercut surfaces 141, the plurality of second concave undercut surfaces 132, and the plurality of second convex undercut surfaces 142 may comprise at least one substantially flat surface.

In some embodiments, when the fastener 100 is viewed in section along a plane intersecting the longitudinal axis 103 of the shaft 105, the first helical thread 110 may comprise a plurality of first bent shapes (comprising at least one surface that is angled relative to the longitudinal axis 103 of the shaft 105 and/or at least one undercut surface) with a plurality of first intermediate portions 151 that are oriented toward (i.e., point toward) the distal end 102 of the shaft 105. This may be referred to as “standard” threading, having a “standard” orientation.

In some embodiments, when the fastener 100 is viewed in section along a plane intersecting the longitudinal axis 103 of the shaft 105, the second helical thread 120 may comprise a plurality of second bent shapes (comprising at least one surface that is angled relative to the longitudinal axis 103 of the shaft 105 and/or at least one undercut surface) with a plurality of second intermediate portions 152 that are oriented toward (i.e., point toward) the proximal end 101 of the shaft 105. This may be referred to as “inverted” threading, having an “inverted” orientation.

In some embodiments, one or more helical threads may morph/transition between a standard orientation and an inverted orientation along a shaft of a fastener.

In some embodiments, at least one of the plurality of first concave undercut surfaces 131, the plurality of first convex undercut surfaces 141, the plurality of second concave undercut surfaces 132, and the plurality of second convex undercut surfaces 142 may comprise at least one curved surface.

As shown in FIG. 1D, the proximally-oriented and distally-oriented surfaces of the first helical thread 110 (i.e., the first concave undercut surfaces 131 and the first convex undercut surfaces 141 in the fastener 100 of FIG. 1D) may not have mirror symmetry relative to each other about any plane perpendicular to the longitudinal axis 103 of the fastener 100. Rather, the first concave undercut surfaces 131 and the first convex undercut surfaces 141 may be generally parallel to each other. The same may be true for the second helical thread 120, in which the second concave undercut surfaces 132 and the second convex undercut surfaces 142 may not have mirror symmetry relative to each other but may be generally parallel to each other.

Conversely, as also shown in FIG. 1D, the proximally-oriented surfaces of the first helical thread 110 may have mirror symmetry relative to the distally-oriented surfaces of the second helical thread 120. Specifically, the first concave undercut surfaces 131 may have mirror symmetry relative to the second convex undercut surfaces 142 about a plane 170 that bisects the space between them and lies perpendicular to the longitudinal axis 103.

Similarly, the distally-oriented surfaces of the first helical thread 110 may have mirror symmetry relative to the proximally-oriented surfaces of the second helical thread 120. Specifically, the second concave undercut surfaces 132 may have mirror symmetry relative to the first convex undercut surfaces 141 about a plane 172 that bisects the space between them and lies perpendicular to the longitudinal axis 103.

This mirror symmetry may be present along most of the length of the first helical thread 110 and the second helical thread 120, with symmetry across different planes arranged between adjacent turns of the first helical thread 110 and the second helical thread 120 along the length of the longitudinal axis 103. Such mirror symmetry may help more effectively capture bone between the first helical thread 110 and the second helical thread 120 and may also facilitate manufacture of the fastener 100.

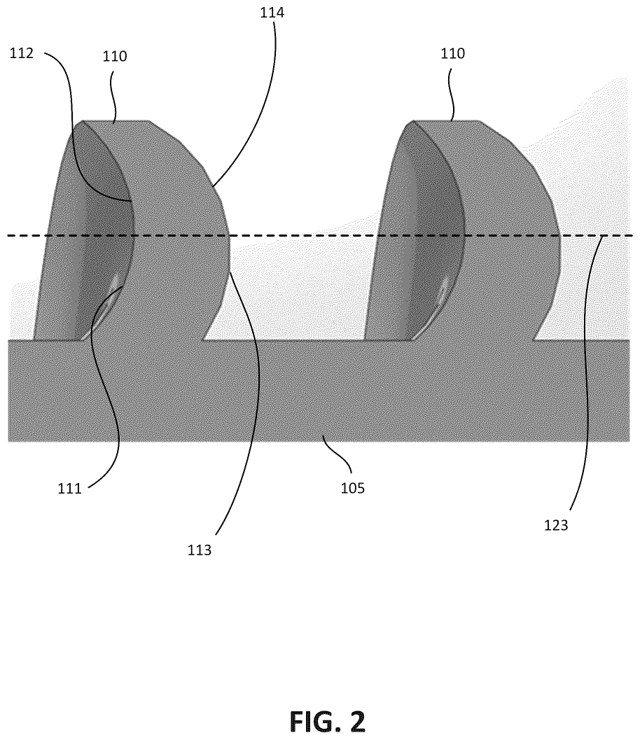

In some embodiments, when the fastener 100 is viewed in section along a plane intersecting the longitudinal axis 103 of the shaft 105, the first helical thread 110 may include at least one partial crescent shape that is oriented toward (i.e., points toward) the distal end 102 of the shaft 105 and/or the proximal end 101 of the shaft 105. FIG. 2 illustrates a partial cross-sectional view of a fastener comprising crescent shapes, as one non-limiting example of such an embodiment.

In some embodiments (not shown), when the fastener 100 is viewed in section along a plane intersecting the longitudinal axis 103 of the shaft 105, the first helical thread 110 may include at least one partial crescent shape that is oriented toward (i.e., points toward) the distal end 102 of the shaft 105, and the second helical thread 120 may include at least one partial crescent shape that is oriented toward (i.e., points toward) the proximal end 101 of the shaft 105.

In some embodiments (not shown), the first helical thread 110 may include a first plurality of partial crescent shapes that are oriented toward (i.e., point toward) the distal end 102 of the shaft 105, and the second helical thread 120 may include a second plurality of partial crescent shapes that are oriented toward (i.e., point toward) the proximal end 101 of the shaft 105.

In some embodiments (not shown), the first plurality of partial crescent shapes and the second plurality of partial crescent shapes may be arranged in alternating succession along the shaft 105 of the fastener 100.

In some embodiments, the first helical thread 110 may be bisected by the line 123 shown in FIG. 2 with each crescent shape including a plurality of first undercut surfaces 111, a plurality of second undercut surfaces 112, a plurality of third undercut surfaces 113, and a plurality of fourth open surfaces 114 similar to the helical threading shown in FIG. 1D, except with curved surfaces in place of flat surfaces.

In some embodiments, the plurality of first undercut surfaces 111 and the plurality of second undercut surfaces 112 may comprise concave curved surfaces. However, it will be understood that portions of the plurality of first undercut surfaces 111 and/or portions of the plurality of second undercut surfaces 112 may also comprise convex curved surfaces and/or flat surfaces (not shown in FIG. 2).

In some embodiments, the plurality of third undercut surfaces 113 and the plurality of fourth open surfaces 114 may comprise convex curved surfaces. However, it will be understood that portions of the plurality of third undercut surfaces 113 and the plurality of fourth open surfaces 114 may also comprise concave curved surfaces and/or flat surfaces (not shown in FIG. 2).

In some embodiments, the plurality of third undercut surfaces 113 and the plurality of fourth open surfaces 114 may be replaced by a ramped surface (such as that utilized in a standard buttress thread design) without any undercuts (not shown in FIG. 2). Likewise, any of the other thread designs disclosed herein may utilize a ramped or buttress thread design on at least one side of the helical thread.

In some embodiments, a fastener may have only standard threads or only inverted threads. The type of threads that are desired may depend on the type and/or magnitude of loads to be applied to the fastener. For example, a screw loaded axially away from the bone in which it is implanted may advantageously have a standard thread, while a screw loaded axially toward the bone in which it is implanted may advantageously have an inverted thread. A screw that may experience multi-axial loading and/or off-loading conditions may advantageously include at least one standard thread and at least one inverted thread in order to increase bone fixation and load sharing between a bone/fastener interface during multi-axial and off-loading conditions to reduce high bone strain and distribute multi-axial forces applied to the bone in a load-sharing, rather than load-bearing, configuration. Shear loads and/or bending moments may also be optimally resisted with any chosen combination of threading, threading morphology, and/or threading variations contemplated herein to optimally resist shear loads, bending moments, multi-axial loading, off-loading conditions, etc.

In some embodiments, fasteners with standard threads may be used in conjunction with fasteners with inverted threads in order to accommodate different loading patterns.

In some embodiments, a single fastener may have both standard and inverted threads, like the fastener 100. Such a combination of threads may help the fastener 100 remain in place with unknown and/or varying loading patterns.

In some embodiments, the geometry of the threading of a fastener (with standard and/or inverted threads) may be varied to suit the fastener for a particular loading scheme. For example, the number of threads, the number of thread starts, the pitch of the threading, the lead(s) of the threading, the shape(s) of the threading, any dimension(s) associated with the threading (e.g., any length(s)/width(s)/height(s)/inflection point(s), etc., associated with the threading), the major diameter(s), the minor diameter(s), any angulation/angles associated with any surfaces of the threading, the “handedness” of the threading (e.g., right-handed vs. left-handed), etc., may be varied accordingly to suit any specific medium of installation, loading pattern, desired radial loading force, pull-out strength, application, procedure, etc., that may be involved.

In some embodiments, the material(s) of any portion of a fastener, bone implant, joint replacement implant, articular member, etc., described herein may include, but are not limited to metals (e.g., titanium, cobalt, stainless steel, etc.), metal alloys, plastics, polymers, ceramics, PEEK, UHMWPE, composites, additive particles, textured surfaces, biologics, biomaterials, bone, etc.

In some embodiments, any of the fasteners, bone implants, joint replacement implants, articular members, etc., described herein may include additional features such as: self-tapping features, locking features (e.g., locking threading formed on a portion of the fastener, such as threading located on or near a head of the fastener), opening(s), cannulation(s), fenestration(s), any style of fastener head (or no fastener head at all), any style of torque connection interface (or no torque connection interface at all), etc.

In some embodiments, any of the fasteners, bone implants, joint replacement implants, articular members, etc., described herein may also include opening(s), cannulation(s), fenestration(s), etc., that may be configured to receive any suitable bone cement or bone augment material therein to facilitate bone in-growth, bone fusion, etc.

In some embodiments, a tap (not shown) may be utilized to pre-form threading in a bone or bone augment material according to any threading shape that is disclosed or contemplated herein. In this manner, taps with any suitable shape may be utilized in conjunction with any fastener described or contemplated herein to match or substantially match the threading geometry of a given fastener or bone implant.

In some embodiments, a minor diameter of the fastener may be selected to match, or substantially match, a diameter of a pilot hole that is formed in a bone to avoid bone blowout when the fastener is inserted into the pilot hole.

Additionally, or alternatively thereto, the type of threads and/or thread geometry may be varied based on the type of bone in which the fastener is to be anchored. For example, fasteners anchored in osteoporotic bone may fare better with standard or inverted threads, or when the pitch, major diameter, and/or minor diameter are increased or decreased, or when the angulation of thread surfaces is adjusted, etc.

In some embodiments, a surgical kit may include one or more fasteners, bone implants, joint replacement implants, articular members, etc., with any of the different thread options described or contemplated herein. The surgeon may select the appropriate components from the kit based on the particular loads to be applied and/or the quality of bone in which the implant(s) are to be anchored.

Continuing with FIG. 1D, in some embodiments the first helical thread 110 may include a plurality of first undercut surfaces 111, a plurality of second undercut surfaces 112, a plurality of third undercut surfaces 113, and a plurality of fourth open surfaces 114.

In some embodiments, the second helical thread 120 may include a plurality of fifth undercut surfaces 125, a plurality of sixth undercut surfaces 126, a plurality of seventh undercut surfaces 127, and a plurality of eighth open surfaces 128.

In some embodiments, one or more of the plurality of first undercut surfaces 111, the plurality of second undercut surfaces 112, the plurality of third undercut surfaces 113, the plurality of fourth open surfaces 114, the plurality of fifth undercut surfaces 125, the plurality of sixth undercut surfaces 126, the plurality of seventh undercut surfaces 127, and the plurality of eighth open surfaces 128 may comprise at least one flat or substantially flat surface.

In some embodiments, the plurality of first undercut surfaces 111, the plurality of third undercut surfaces 113, the plurality of sixth undercut surfaces 126, and the plurality of eighth open surfaces 128 may be angled towards the distal end 102 of the shaft 105.

In some embodiments, the plurality of second undercut surfaces 112, the plurality of fourth open surfaces 114, the plurality of fifth undercut surfaces 125, and the plurality of seventh undercut surfaces 127 may be angled towards the proximal end 101 of the shaft 105.

In some embodiments, when the fastener 100 is viewed in section along a plane that intersects the longitudinal axis 103 of the shaft 105 (as shown in FIG. 1D), the first helical thread 110 may include at least one chevron shape that is oriented toward (i.e., points toward) the distal end 102 of the shaft 105. Likewise, the second helical thread 120 may also include at least one chevron shape that is oriented toward (i.e., points toward) the proximal end 101 of the shaft 105.

In some embodiments, when the fastener 100 is viewed in section along a plane that intersects the longitudinal axis 103 of the shaft 105 (as shown in FIG. 1D), the first helical thread 110 may include a first plurality of chevron shapes that are oriented toward (i.e., point toward) the distal end 102 of the shaft 105. Likewise, the second helical thread 120 may include a second plurality of chevron shapes that are oriented toward (i.e., point toward) the proximal end 101 of the shaft 105.

In some embodiments, the first plurality of chevron shapes and the second plurality of chevron shapes may be arranged in alternating succession along the shaft 105 of the fastener 100, (e.g., see FIG. 1D).

In some embodiments, a plurality of first interlocking spaces 161 and a plurality of second interlocking spaces 162 may be formed between the first helical thread 110 and the second helical thread 120 along the shaft 105 of the fastener 100.

In some embodiments, the plurality of first interlocking spaces 161 may be formed intermediate the first concave undercut surfaces 131 and the second concave undercut surfaces 132.

In some embodiments, the plurality of second interlocking spaces 162 may be formed intermediate the first convex undercut surfaces 141 and the second convex undercut surfaces 142.

In some embodiments, the plurality of first interlocking spaces 161 may be larger in size than the plurality of second interlocking spaces.

In some embodiments, the plurality of first interlocking spaces 161 and the plurality of second interlocking spaces 162 may be shaped and/or configured to interlock with bone/other tissues received therein to increase fixation of the fastener 100 within the bone/other tissues and provide additional resistance against multi-axial forces that may be applied to the fastener 100 and/or the bone/other tissues.

In some embodiments, the plurality of second undercut surfaces 112 and the plurality of sixth undercut surfaces 126 may be angled toward each other to trap bone/bone augment material within the plurality of first interlocking spaces 161 in order to increase fixation and resistance against multi-axial forces.

In some embodiments, the plurality of third undercut surfaces 113 and the plurality of seventh undercut surfaces 127 may be angled toward each other to trap bone/other tissues within the plurality of second interlocking spaces 162 in order to increase fixation and resistance against multi-axial forces.

In some embodiments, the plurality of first undercut surfaces 111 and the plurality of fifth undercut surfaces 125 may each form an angle α with respect to the longitudinal axis 103 of the shaft 105, as shown in FIG. 1D.

In some embodiments, the angle α may be greater than 90 degrees.

In some embodiments, the plurality of second undercut surfaces 112 and the plurality of sixth undercut surfaces 126 may each form an angle β with respect to the longitudinal axis 103 of the shaft 105.

In some embodiments, the angle θ may be less than 90 degrees.

In some embodiments, the plurality of third undercut surfaces 113 and the plurality of seventh undercut surfaces 127 may each form an angle θ with respect to the longitudinal axis 103 of the shaft 105.

In some embodiments, the angle θ may be approximately 90 degrees.

In some embodiments, the angle θ may be greater than 90 degrees.

It will be understood that any fastener, bone implant, joint replacement implant, articular member, etc., described or contemplated herein may include any thread configuration, feature, or morphology that is described or contemplated herein to achieve optimal fixation within a given bone/tissue. Moreover, it will also be understood that any fastener, bone implant, joint replacement implant, articular member, etc., described or contemplated herein may be utilized in conjunction with (or within) any system, method, or instrumentation that is described or contemplated herein.

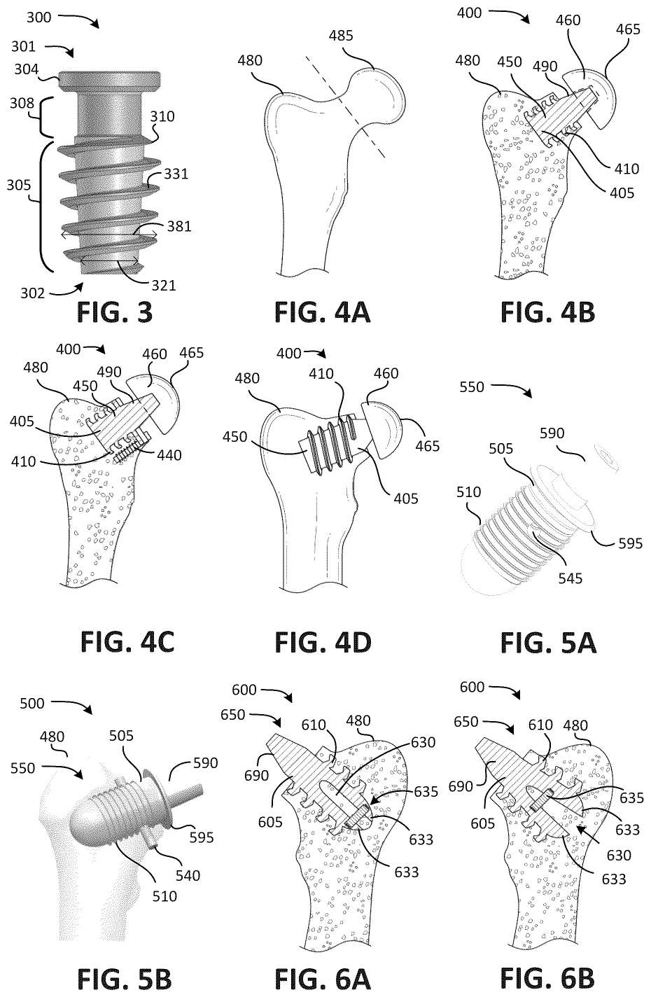

FIG. 3 illustrates a tapered fastener, tapered joint replacement implant, or tapered bone implant 300, according to an embodiment of the present disclosure. The morphology of the tapered bone implant 300 may provide increased fixation by applying compression forces on a bone as the tapered bone implant 300 is inserted into the bone (e.g., by compressing cancellous/subchondral bone during insertion of the tapered bone implant 300 into an intramedullary canal of a bone, as one non-limiting example).

In some embodiments, the tapered bone implant 300 may generally include a proximal end 301, a distal end 302, a tapered shaft portion 305, a neck portion 308, a head 304, a torque connection interface (not shown) that may be formed in/on the head 304, and at least one tapered helical thread 310 disposed about the tapered shaft portion 305.

In some embodiments, the tapered shaft portion 305 and/or the at least one tapered helical thread 310 may comprise one or more self-tapping features or cutting flutes (not shown).

In some embodiments, the tapered shaft portion 305 may have a continuously variable minor diameter 321 generally defined by the shape of the tapered shaft portion 305, and a continuously variable major diameter 381 generally defined by the shape of the at least one tapered helical thread 310 disposed about the tapered shaft portion 305.

In some embodiments, the continuously variable minor diameter 321 defined by the shape of the tapered shaft portion 305 may comprise an at least partially conical shape.

In some embodiments, the continuously variable major diameter 381 defined by the shape of the at least one tapered helical thread 310 disposed about the tapered shaft portion 305 may comprise an at least partially conical shape.

In some embodiments, the continuously variable minor diameter 321 defined by the shape of the tapered shaft portion 305 may generally decrease moving from the proximal end 301 toward the distal end 302 of the tapered bone implant 300.

In some embodiments, the continuously variable major diameter 381 defined by the shape of the at least one tapered helical thread 310 disposed about the tapered shaft portion 305 may generally decrease moving from the proximal end 301 toward the distal end 302 of the tapered bone implant 300.

In some embodiments, the at least one tapered helical thread 310 may include at least one concave undercut surface 331 that may be angled towards one of the proximal end 301 and the distal end 302 of the tapered bone implant 300. However, it will also be understood that the tapered bone implant 300 may include any thread configuration, feature, or morphology described or contemplated herein with respect to any fastener/implant to achieve optimal fixation within a given bone, tissue, bone cement, bone augment material, etc. For example, in some embodiments the at least one tapered helical thread 310 may comprise standard or inverted threading, a “dual start” thread configuration, crescent shapes, etc.

Moreover, it will also be understood that the tapered bone implant 300, or portions of the general design/shape of the tapered bone implant 300, may be utilized in conjunction with (or within) any fastener, bone implant, system, procedure, or instrumentation that is described or contemplated herein. For example, any of the fasteners, bone implants, joint replacement implants, etc., that are described or contemplated herein may generally be configured to include a tapered shaft portion with tapered helical threading disposed about the tapered shaft portion.

FIGS. 4A-4D illustrate various views of a joint replacement implant system 400 for a proximal femur 480 and/or a femoral head 485, according to embodiments of the present disclosure.

In some embodiments, the joint replacement implant system 400 may include a fastener, bone implant, or joint replacement implant 450, as well as an articular member 460 disposed at a proximal end of the joint replacement implant 450 comprising an artificial articular surface or articular surface 465.

In some embodiments, the joint replacement implant 450 may include a shaft 405 having a proximal end, a distal end, a threaded shaft portion, and a helical thread 410 disposed about the shaft 405 defining a length of the threaded shaft portion.

In some embodiments, the shaft 405 and/or the helical thread 410 may also include one or more self-tapping features or cutting flutes (not shown).

In some embodiments, the helical thread 410 may include a concave undercut surface angled towards one of the proximal end and the distal end of the shaft 405. However, it will also be understood that the joint replacement implant 450 may (or may not) include any thread configuration, feature, or morphology described or contemplated herein with respect to any fastener, bone implant, or joint replacement implant to achieve optimal fixation within a given bone, tissue, bone cement, bone augment material, etc. For example, in some embodiments the helical thread 410 may comprise standard or inverted threading, a “dual start” thread configuration, tapered helical threading, crescent shapes, etc. Moreover, it will also be understood that the joint replacement implant 450 may be utilized in conjunction with (or within) any system, method, procedure, or instrumentation described or contemplated herein.

In some embodiments, the joint replacement implant system 400 may also include one or more fasteners 440 configured to help provide additional stabilization for the joint replacement implant 450 within the proximal femur 480, as shown in FIG. 4C.

In some embodiments, the joint replacement implant system 400 may be utilized with bone cement, any type of bone augment material(s), any feature(s) that may facilitate bony ingrowth, etc., to provide additional stabilization for the joint replacement implant 450.

It will be understood that the one or more fasteners 440 may (or may not) include any thread configuration, feature, or morphology described or contemplated herein with respect to any fastener, bone implant, or joint replacement implant to achieve optimal fixation within a given bone, tissue, bone cement, bone augment material, etc. Moreover, it will also be understood that the one or more fasteners 440 may be utilized in conjunction with (or within) any system, method, procedure, or instrumentation described or contemplated herein.

In some embodiments, the articular member 460 may comprise a femoral head prosthesis configured to replace the femoral head 485 after it has been resected, as shown in FIG. 4A.

In some embodiments, the articular surface 465 of the articular member 460 may comprise a convex (or concave) semi-spherical articular surface.

In some embodiments, the articular member 460 may be removably couplable with the joint replacement implant 450.

In some embodiments, the articular member 460 may be integrally formed with the joint replacement implant 450.

In some embodiments, the joint replacement implant 450 may comprise a single fixed stem component that is couplable with the articular member 460.

In some embodiments, the joint replacement implant system 400 the joint replacement implant 450 may be couplable to the articular member 460 via an attachment feature 490 at the proximal end of the joint replacement implant 450.

In some embodiments, the attachment feature 490 may comprise a post. In some embodiments, the post may comprise a Morse taper. However, it will also be understood that the attachment feature 490 may comprise any suitable structure for coupling the articular member 460 to the joint replacement implant 450 including, but not limited to: a snap-fit feature, a tab feature, a dovetail feature, a sleeve-in-sleeve feature design, etc.

In some embodiments, the joint replacement implant 450 may comprise a modular stem system comprising one or more intermediary components or one or more proximal femur intermediary components (not shown), and the articular member 460 may be couplable with at least one of the one or more intermediary components to dispose the articular member 460 at the proximal end of the joint replacement implant 450.

In some embodiments, the one or more intermediary components may include one of: a Morse taper feature, a snap-fit feature, a tab feature, a dovetail feature, a sleeve-in-sleeve feature, etc., intermediate the joint replacement implant 450 and the articular member 460.

In some embodiments, the one or more intermediary components may (or may not) include any thread configuration, feature, or morphology described or contemplated herein with respect to any fastener, bone implant, or joint replacement implant to couple or dispose the articular member 460 at the proximal end of the joint replacement implant 450.

In some embodiments, a method for implanting the joint replacement implant system 400 shown in FIGS. 4A-4D may include: resecting the femoral head 485 from the proximal femur 480 (see FIG. 4A); preparing, forming, or drilling a bone tunnel into the bone (e.g., a neck of the proximal femur 480) via a drill, a broach, a punch, etc., (not shown); tapping a helical bone thread about the bone tunnel with a tapping tool (not shown), or utilizing self-tapping features on the joint replacement implant 450 to form the helical bone thread about the bone tunnel; inserting the joint replacement implant 450 into the prepared bone tunnel by rotating the joint replacement implant relative to the bone tunnel to insert the helical thread into the helical bone thread (or form the helical bone thread about the bone tunnel via the one or more self-tapping features) and insert the joint replacement implant into the bone tunnel; and coupling the articular member 460 to the proximal end of the joint replacement implant 450 (e.g., via the attachment feature 490, etc.).

FIGS. 5A and 5B illustrate various views of a joint replacement implant system 500 for the proximal femur 480, according to another embodiment of the present disclosure.

In some embodiments, the joint replacement implant system 500 may include a joint replacement implant 550, which may be couplable to the articular member 460 via an attachment feature 590 at the proximal end of the joint replacement implant 550.

In some embodiments, the attachment feature 590 may comprise a post. In some embodiments, the post may comprise a Morse taper. However, it will also be understood that the attachment feature 590 may comprise any suitable structure for coupling the articular member 460 to the joint replacement implant 550 including, but not limited to: a snap-fit feature, a tab feature, a dovetail feature, a sleeve-in-sleeve feature design, etc.

In some embodiments, the joint replacement implant 550 may include a shaft 505 having a proximal end, a distal end, a threaded shaft portion, and a helical thread 510 disposed about the shaft 505 defining a length of the threaded shaft portion.

In some embodiments, the shaft 505 may also include one or more transverse passageways 545 formed therein and configured to receive one or more or pins or fasteners 540 therethrough to provide additional stabilization for the joint replacement implant 550, as shown in FIG. 5B. It will be understood that the one or more pins or fasteners 540 may (or may not) include any thread configuration, feature, or morphology described or contemplated herein with respect to any fastener, bone implant, or joint replacement implant to achieve optimal fixation within a given bone, tissue, bone cement, bone augment material, etc. Moreover, it will also be understood that the one or more pins or fasteners 540 may be utilized in conjunction with (or within) any system, method, procedure, or instrumentation described or contemplated herein.

In some embodiments, the distal end of the shaft 505 may be rounded or may comprise a hemi-spherical shape.

In some embodiments, the joint replacement implant 550 may include a flange component 595 intermediate the attachment feature 590 and the shaft 505.

In some embodiments, the shaft 505 and/or the helical thread 510 may also include one or more self-tapping features or cutting flutes (not shown).

In some embodiments, the helical thread 510 may include a concave undercut surface angled towards one of the proximal end and the distal end of the shaft 505. However, it will also be understood that the joint replacement implant 550 may (or may not) include any thread configuration, feature, or morphology described or contemplated herein with respect to any fastener, bone implant, or joint replacement implant to achieve optimal fixation within a given bone, tissue, bone cement, bone augment material, etc. For example, in some embodiments the helical thread 510 may comprise standard or inverted threading, a “dual start” thread configuration, tapered helical threading, crescent shapes, etc. Moreover, it will also be understood that the joint replacement implant 550 may be utilized in conjunction with (or within) any system, method, procedure, or instrumentation described or contemplated herein.

In some embodiments, the joint replacement implant system 500 may be utilized with bone cement, any type of bone augment material(s), any feature(s) that may facilitate bony ingrowth, etc., to provide additional stabilization for the joint replacement implant 550.

In some embodiments, the articular member 460 may comprise a femoral head prosthesis configured to replace the femoral head 485 after it has been resected.

In some embodiments, the articular surface 465 of the articular member 460 may comprise a convex (or concave) semi-spherical articular surface.

In some embodiments, the articular member 460 may be removably couplable with the joint replacement implant 550.

In some embodiments, the articular member 460 may be integrally formed with the joint replacement implant 550.

In some embodiments, the joint replacement implant 550 may comprise a single fixed stem component that is couplable with the articular member 460.

In some embodiments, the joint replacement implant 550 may comprise a modular stem system comprising one or more intermediary components or one or more proximal femur intermediary components (not shown), and the articular member 460 may be couplable with at least one of the one or more intermediary components to couplably dispose the articular member 460 near the proximal end of the joint replacement implant 550.

In some embodiments, the one or more intermediary components may include one of: a Morse taper feature, a snap-fit feature, a tab feature, a dovetail feature, a sleeve-in-sleeve feature, etc., intermediate the joint replacement implant 550 and the articular member 460.

In some embodiments, the one or more intermediary components may (or may not) include any thread configuration, feature, or morphology described or contemplated herein with respect to any fastener, bone implant, or joint replacement implant to couple or dispose the articular member 460 at the proximal end of the joint replacement implant 550.

In some embodiments, a method for implanting the joint replacement implant system 500 shown in FIGS. 5A and 5B may include: resecting the femoral head 485 from the proximal femur 480; drilling a bone tunnel into a neck of the proximal femur 480 with a drill tool (not shown); tapping a helical bone thread about the bone tunnel with a tapping tool (not shown), or utilizing self-tapping features on the joint replacement implant 550 to form the helical bone thread about the bone tunnel as the joint replacement implant 550 is rotated into the bone tunnel; inserting the joint replacement implant 550 into the prepared bone tunnel; and coupling the articular member 460 to the proximal end of the joint replacement implant 550.

FIGS. 6A and 6B illustrate various views of a joint replacement implant system 600 for the proximal femur 480, according to another embodiment of the present disclosure.

In some embodiments, the joint replacement implant system 600 may include a joint replacement implant 650, which may be coupled to the articular member 460 via an attachment feature 690 at the proximal end of the joint replacement implant 650.

In some embodiments, the attachment feature 690 may comprise a post. In some embodiments, the post may comprise a Morse taper. However, it will also be understood that the attachment feature 690 may comprise any suitable structure for coupling the articular member 460 to the joint replacement implant 650 including, but not limited to: a snap-fit feature, a tab feature, a dovetail feature, a sleeve-in-sleeve feature design, etc.

In some embodiments, the joint replacement implant 650 may include a shaft 605 having a proximal end, a distal end, a threaded shaft portion, and a helical thread 610 disposed about the shaft 605 defining a length of the threaded shaft portion.

In some embodiments, the distal end of the shaft 605 may be rounded or may comprise a hemi-spherical shape.

In some embodiments, the shaft 605 and/or the helical thread 610 may also include one or more self-tapping features or cutting flutes (not shown).

In some embodiments, the helical thread 610 may include a concave undercut surface angled towards one of the proximal end and the distal end of the shaft 605. However, it will also be understood that the joint replacement implant 650 may (or may not) include any thread configuration, feature, or morphology described or contemplated herein with respect to any fastener, bone implant, or joint replacement implant to achieve optimal fixation within a given bone, tissue, bone cement, bone augment material, etc. For example, in some embodiments the helical thread 610 may comprise standard or inverted threading, a “dual start” thread configuration, tapered helical threading, crescent shapes, etc. Moreover, it will also be understood that the joint replacement implant 650 may be utilized in conjunction with (or within) any system, method, procedure, or instrumentation described or contemplated herein.

In some embodiments, the joint replacement implant system 600 may be utilized with bone cement, any type of bone augment material(s), any feature(s) that may facilitate bony ingrowth, etc., to provide additional stabilization for the joint replacement implant 650.

In some embodiments, the articular member 460 may comprise a femoral head prosthesis configured to replace the femoral head 485 after it has been resected.

In some embodiments, the articular surface 465 of the articular member 460 may comprise a convex (or concave) semi-spherical articular surface.

In some embodiments, the articular member 460 may be removably couplable with the joint replacement implant 650.

In some embodiments, the articular member 460 may be integrally formed with the joint replacement implant 650.

In some embodiments, the joint replacement implant 650 may comprise a single fixed stem component that is couplable with the articular member 460.

In some embodiments, the joint replacement implant 650 may comprise a modular stem system comprising one or more intermediary components or one or more proximal femur intermediary components (not shown), and the articular member 460 may be couplable with at least one of the one or more intermediary components to dispose the articular member 460 at or near the proximal end of the joint replacement implant 650.

In some embodiments, the one or more intermediary components may include one of: a Morse taper feature, a snap-fit feature, a tab feature, a dovetail feature, a sleeve-in-sleeve feature, etc., intermediate the joint replacement implant 650 and the articular member 460.

In some embodiments, the one or more intermediary components may (or may not) include any thread configuration, feature, or morphology described or contemplated herein with respect to any fastener, bone implant, or joint replacement implant to couple or dispose the articular member 460 at the proximal end of the joint replacement implant 650.

In some embodiments, the joint replacement implant 650 may include one or more splitting slots 630 longitudinally formed in the shaft 605 to define a plurality of legs 633 in the shaft 605 of the joint replacement implant 650.

In some embodiments, the joint replacement implant 650 may be formed of a flexible or semi-flexible material to allow the plurality of legs 633 to come together or move toward each other in a first contracted state (e.g., see FIG. 6A), and/or spread apart or move away from each other into a second expanded state (e.g., see FIG. 6B) to provide additional fixation/stabilization for the joint replacement implant 650.

In some embodiments, the joint replacement implant system 600 may include an actuation member 635 configured to selectively deploy the plurality of legs 633 between the first contracted state and the second expanded state.

In some embodiments, the actuation member 635 may comprise a plug or wedge shaped member (not shown) which may be pulled proximally and/or moved distally to selectively deploy the plurality of legs 633 between the first contracted state and the second expanded state.

In some embodiments, the actuation member 635 may comprise a threaded set screw that may be actuated with a driver tool (not shown) placed through a central longitudinal passageway formed in the shaft 605 and coupled to the threaded set screw. In this manner, the threaded set screw may be rotated in a first direction to translate the threaded set screw in a first distal direction within the joint replacement implant 650 to cause the plurality of legs 633 to move toward each other into the first contracted state, and the threaded set screw may be rotated in a second direction to translate the threaded set screw in a second proximal direction within the joint replacement implant 650 to cause the plurality of legs 633 to move away from each other into the second expanded state.

In some embodiments, a method for implanting the joint replacement implant system 600 shown in FIGS. 6A and 6B may include: resecting the femoral head 485 from the proximal femur 480; drilling a bone tunnel into a neck of the proximal femur 480 with a drill tool (not shown); tapping a helical bone thread about the bone tunnel with a tapping tool (not shown), or utilizing self-tapping features on the joint replacement implant 650 to form the helical bone thread about the bone tunnel during insertion; inserting the joint replacement implant 650 into the prepared bone tunnel; actuating the actuation member 635 to deploy the plurality of legs 633 into the expanded state; and coupling the articular member 460 to the proximal end of the joint replacement implant 650. Some embodiments of the method may also include backing out the joint replacement implant 650 by a small amount after the plurality of legs 633 have been deployed into the expanded state to create an even tighter interference fit between the expanded joint replacement implant 650 and the proximal femur 480.

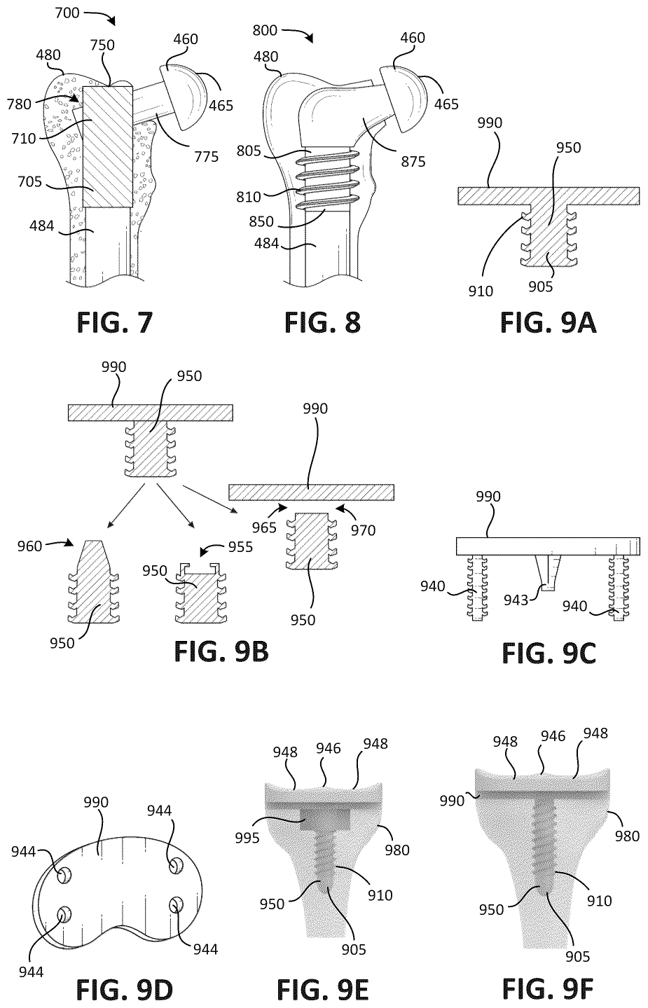

FIG. 7 illustrates a joint replacement implant system 700 for the proximal femur 480, according to another embodiment of the present disclosure.

In some embodiments, the joint replacement implant system 700 may generally include a joint replacement implant 750 couplable within an intramedullary canal 484 of the proximal femur 480, an intermediary neck component 775 coupled to the joint replacement implant 750, and the articular member 460 coupled to a proximal end of the intermediary neck component 775.

In some embodiments, the intermediary neck component 775 may be coupled to an intermediate or proximal portion of the joint replacement implant 750.

In some embodiments, a distal end of the intermediary neck component 775 may be received within a recess or passageway 780 formed through the intermediate or proximal portion of the joint replacement implant 750 to couple the intermediary neck component 775 to the joint replacement implant 750.

In some embodiments, the intermediary neck component 775 may comprise an attachment feature (not shown) to couple the articular member 460 to the intermediary neck component 775. In some embodiments, the attachment feature may include at least one of: a Morse taper feature, a snap-fit feature, a tab feature, a dovetail feature, a sleeve-in-sleeve feature, etc., between the intermediary neck component 775 and the articular member 460.

In some embodiments, the joint replacement implant 750 may include a shaft 705 having a proximal end, a distal end, a threaded shaft portion, and a helical thread 710 disposed about the shaft 705 defining a length of the threaded shaft portion.

In some embodiments, the shaft 705 and/or the helical thread 710 may include one or more self-tapping features or cutting flutes (not shown).

In some embodiments, the helical thread 710 may include a concave undercut surface angled towards one of the proximal end and the distal end of the shaft 705. However, it will also be understood that the joint replacement implant 750 may (or may not) include any thread configuration, feature, or morphology described or contemplated herein with respect to any fastener, bone implant, or joint replacement implant to achieve optimal fixation within a given bone, tissue, bone cement, bone augment material, etc. For example, in some embodiments the helical thread 710 may comprise standard or inverted threading, a “dual start” thread configuration, tapered helical threading, crescent shapes, etc. Moreover, it will also be understood that the joint replacement implant 750 may be utilized in conjunction with (or within) any system, method, procedure, or instrumentation described or contemplated herein.

In some embodiments, the joint replacement implant system 700 may be utilized with bone cement, any type of bone augment material(s), any feature(s) that may facilitate bony ingrowth, one or more additional pins or fasteners, etc., to provide additional stabilization for the joint replacement implant 750 within the intramedullary canal 484 of the proximal femur 480.

In some embodiments, the articular member 460 may comprise a femoral head prosthesis configured to replace the femoral head 485 after it has been resected.

In some embodiments, the articular surface 465 of the articular member 460 may comprise a convex (or concave) semi-spherical articular surface.

In some embodiments, the articular member 460 may be removably couplable with the intermediary neck component 775.

In some embodiments, the articular member 460 may be integrally formed with the intermediary neck component 775.

In some embodiments, the joint replacement implant 750 may comprise a modular stem system comprising one or more intermediary components or one or more proximal femur intermediary components (not shown), and the articular member 460 may be couplable with at least one of the one or more intermediary components to couple the articular member 460 to the joint replacement implant 750.

In some embodiments, the one or more intermediary components may comprise the intermediary neck component 775.

In some embodiments, the one or more intermediary components may include one of: a Morse taper feature, a snap-fit feature, a tab feature, a dovetail feature, a sleeve-in-sleeve feature, etc., to couple intermediate the articular member 460 and the joint replacement implant 750.

In some embodiments, the one or more intermediary components may (or may not) include any thread configuration, feature, or morphology described or contemplated herein with respect to any fastener, bone implant, or joint replacement implant to facilitate coupling of the articular member 460 with the joint replacement implant 750.

In some embodiments, a method for implanting the joint replacement implant system 700 shown in FIG. 7 may include: resecting the femoral head 485 from the proximal femur 480; drilling, reaming, or broaching a bone tunnel into the intramedullary canal 484 of the proximal femur 480 with a suitable tool (not shown); tapping a helical bone thread about the bone tunnel with a tapping tool (not shown), or utilizing self-tapping features on the joint replacement implant 750 to form the helical bone thread about the bone tunnel during insertion; inserting the joint replacement implant 750 into the prepared bone tunnel within the intramedullary canal 484; coupling the distal end of the intermediary neck component 775 to the joint replacement implant 750; and coupling the articular member 460 to the proximal end of intermediary neck component 775.

FIG. 8 illustrates a joint replacement implant system 800 for the proximal femur 480, according to another embodiment of the present disclosure.

In some embodiments, the joint replacement implant system 800 may generally include a joint replacement implant 850 couplable within an intramedullary canal 484 of the proximal femur 480, an intermediary neck component 875 coupled to the joint replacement implant 850, and the articular member 460 coupled to a proximal end of the intermediary neck component 875.

In some embodiments, a distal end of the intermediary neck component 875 may be coupled to a proximal end of the joint replacement implant 850.

In some embodiments, a distal end of the intermediary neck component 875 may be coupled within a central longitudinal passageway formed in the joint replacement implant 850.

In some embodiments, the intermediary neck component 875 may be integrally formed with the joint replacement implant 850.

In some embodiments, the intermediary neck component 875 may comprise an attachment feature (not shown) to couple the articular member 460 to the intermediary neck component 875. In some embodiments, the attachment feature may include one of: a Morse taper feature, a snap-fit feature, a tab feature, a dovetail feature, a sleeve-in-sleeve feature, etc., between the intermediary neck component 875 and the articular member 460.

In some embodiments, the joint replacement implant 850 may include a shaft 805 having a proximal end, a distal end, a threaded shaft portion, and a helical thread 810 disposed about the shaft 805 defining a length of the threaded shaft portion.

In some embodiments, the shaft 805 and/or the helical thread 810 may include one or more self-tapping features or cutting flutes (not shown).

In some embodiments, the helical thread 810 may include a concave undercut surface angled towards one of the proximal end and the distal end of the shaft 805. However, it will also be understood that the joint replacement implant 850 may (or may not) include any thread configuration, feature, or morphology described or contemplated herein with respect to any fastener, bone implant, or joint replacement implant to achieve optimal fixation within a given bone, tissue, bone cement, bone augment material, etc. For example, in some embodiments the helical thread 810 may comprise standard or inverted threading, a “dual start” thread configuration, tapered helical threading, crescent shapes, etc. Moreover, it will also be understood that the joint replacement implant 850 may be utilized in conjunction with (or within) any system, method, procedure, or instrumentation described or contemplated herein.

In some embodiments, the joint replacement implant system 800 may be utilized with bone cement, any type of bone augment material(s), any feature(s) that may facilitate bony ingrowth, one or more additional pins or fasteners, etc., to provide additional stabilization for the joint replacement implant 850 within the intramedullary canal 484 of the proximal femur 480.

In some embodiments, the articular member 460 may comprise a femoral head prosthesis configured to replace the femoral head 485 after it has been resected.

In some embodiments, the articular surface 465 of the articular member 460 may comprise a convex (or concave) semi-spherical articular surface.

In some embodiments, the articular member 460 may be removably couplable with the intermediary neck component 875.

In some embodiments, the articular member 460 may be integrally formed with the intermediary neck component 875.

In some embodiments, the joint replacement implant 850 may comprise a modular stem system comprising one or more intermediary components or one or more proximal femur intermediary components (not shown), and the articular member 460 may be couplable with at least one of the one or more intermediary components to couple the articular member 460 to the joint replacement implant 850.

In some embodiments, the one or more intermediary components may comprise the intermediary neck component 875.

In some embodiments, the one or more intermediary components may include one of: a Morse taper feature, a snap-fit feature, a tab feature, a dovetail feature, a sleeve-in-sleeve feature, etc., to couple the articular member 460 to the joint replacement implant 850.

In some embodiments, the one or more intermediary components may (or may not) include any thread configuration, feature, or morphology described or contemplated herein with respect to any fastener, bone implant, or joint replacement implant to facilitate coupling of the articular member 460 with the joint replacement implant 850.

In some embodiments, a method for implanting the joint replacement implant system 800 shown in FIG. 8 may include: resecting the femoral head 485 from the proximal femur 480; drilling, reaming, or broaching a bone tunnel into the intramedullary canal 484 of the proximal femur 480 with a suitable tool (not shown); tapping a helical bone thread about the bone tunnel with a tapping tool (not shown), or utilizing self-tapping features on the joint replacement implant 850 to form the helical bone thread about the bone tunnel during insertion; inserting the joint replacement implant 850 into the prepared bone tunnel within the intramedullary canal 484; coupling the distal end of the intermediary neck component 875 to the joint replacement implant 850; and coupling the articular member 460 to the proximal end of intermediary neck component 875.

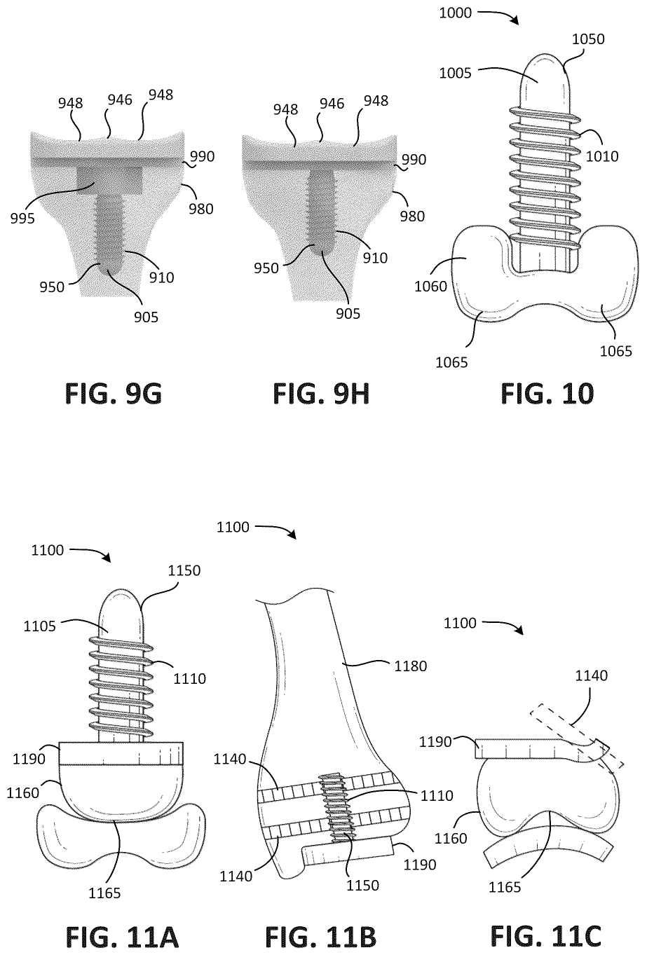

FIGS. 9A-9H illustrate various tibial knee joint implants of one or more knee joint replacement implant systems for a proximal tibia 980, according to embodiments of the present disclosure.

In some embodiments, the joint replacement implant systems may include a joint replacement implant 950, which may be coupled to an articular member 946 via an attachment feature 990 disposed at a proximal end of the joint replacement implant 950.

In some embodiments, the articular member 946 may be removably couplable with the attachment feature 990 and/or the joint replacement implant 950.

In some embodiments, the articular member 946 may be integrally formed with the attachment feature 990 and/or the joint replacement implant 950.

In some embodiments, the articular member 946 may comprise one or more artificial articular surfaces or articular surface 948.

In some embodiments, the articular member 946 may comprise a tibial insert prosthesis.

In some embodiments, the articular surface 948 may comprise one or more concave (or convex) articular surfaces formed in the tibial insert prosthesis.

In some embodiments, the attachment feature 990 may comprise a tibial tray, proximal tibial tray, tray feature, or intermediary tray component configured to receive the articular member 946 or tibial insert prosthesis therein to couple the articular member 946 to the joint replacement implant 950.

In some embodiments, the joint replacement implant 950 may include a shaft 905 having a proximal end, a distal end, a threaded shaft portion, and a helical thread 910 disposed about the shaft 905 defining a length of the threaded shaft portion.

In some embodiments, the shaft 905 and/or the helical thread 910 may also include one or more self-tapping features or cutting flutes (not shown).

In some embodiments, the joint replacement implant 950 may include a flange component 995 intermediate the attachment feature 990 and the shaft 905.

In some embodiments, the helical thread 910 may include a concave undercut surface angled towards one of the proximal end and the distal end of the shaft 905. However, it will also be understood that the joint replacement implant 950 may (or may not) include any thread configuration, feature, or morphology described or contemplated herein with respect to any fastener, bone implant, or joint replacement implant to achieve optimal fixation within a given bone, tissue, bone cement, bone augment material, etc. For example, in some embodiments the helical thread 910 may comprise standard or inverted threading, a “dual start” thread configuration, tapered helical threading, crescent shapes, etc. Moreover, it will also be understood that the joint replacement implant 950 may be utilized in conjunction with (or within) any system, method, procedure, or instrumentation described or contemplated herein.

In some embodiments, the joint replacement implant system may be utilized with bone cement, any type of bone augment material(s), any feature(s) that may facilitate bony ingrowth, etc., to provide additional stabilization for the joint replacement implant 950.

In some embodiments, the joint replacement implant 950 may comprise a stem component that is integrally formed with the attachment feature 990 and/or the articular member 946, as shown in FIG. 9A.

In some embodiments, the joint replacement implant 950 may comprise a modular stem system comprising one or more intermediary components or one or more proximal tibia intermediary components, as shown in FIG. 9B. In these embodiments, the attachment feature 990 may be couplable with the joint replacement implant 950 via at least one of the one or more intermediary components.

In some embodiments, the one or more intermediary components may include one of: a Morse taper feature 955, a dovetail feature 960, a snap-fit feature 965, a tab feature 970, a sleeve-in-sleeve feature, etc., as illustrated in the non-limiting examples shown in FIG. 9B.

In some embodiments, the one or more intermediary components may include the attachment feature 990, tibial tray, proximal tibial tray, tray feature, or intermediary tray component.

In some embodiments, the one or more intermediary components may (or may not) include any thread configuration, feature, or morphology described or contemplated herein with respect to any fastener, bone implant, or joint replacement implant.

In some embodiments, the joint replacement implant system may also include one or more fasteners 940 and/or one or more keels 943, as shown in FIG. 9C, to help stabilize the joint replacement implant system within a proximal tibia.

In some embodiments, the one or more keels 943 may be integrally formed with the attachment feature 990.

In some embodiments, the one or more fasteners 940 and/or the one or more keels 943 may be received through one or more recesses or passageways 944. FIG. 9D shows a top view of the attachment feature 990 with one or more recesses or passageways 944 formed therethrough. In this manner, the attachment feature 990 may be coupled to the one or more fasteners 940, the one or more keels 943, and/or the joint replacement implant 950.

In some embodiments, the joint replacement implant system may be utilized with bone cement, any type of bone augment material(s), any feature(s) that may facilitate bony ingrowth, etc., to provide additional stabilization for the joint replacement implant 950, the one or more fasteners 940, and/or the one or more keels 943. It will also be understood that the one or more fasteners 940 and/or the one or more keels 943 may (or may not) include any thread configuration, feature, or morphology described or contemplated herein with respect to any fastener, bone implant, or joint replacement implant to achieve optimal fixation within a given bone, tissue, bone cement, bone augment material, etc. Moreover, it will also be understood that the one or more fasteners 940 and/or the one or more keels 943 may be utilized in conjunction with (or within) any system, method, procedure, or instrumentation described or contemplated herein.

In some embodiments, methods for implanting the joint replacement implant systems shown in FIGS. 9A-9H may generally include: resecting a portion of the proximal tibia 980 and/or forming a bone tunnel into the proximal tibia 980 with a saw tool, a bone remover tool, a reamer tool, a broach tool, a drill tool, etc., (not shown); tapping a helical bone thread about the bone tunnel with a tapping tool (not shown), or utilizing self-tapping features on the joint replacement implant 950 to form the helical bone thread about the bone tunnel during insertion; inserting the joint replacement implant 950 into the prepared bone tunnel (and/or inserting one or more fasteners/keels into the proximal tibia 980, etc.); and coupling the joint replacement implant 950 to the articular member 946 and/or the attachment feature 990 (if needed).

FIG. 10 illustrates a joint replacement implant system 1000 for a distal femur (not shown), according to an embodiment of the present disclosure.

In some embodiments, the joint replacement implant system 1000 may include a joint replacement implant 1050 couplable with an articular member 1060 at the proximal end of the joint replacement implant 1050.

In some embodiments, the articular member 1060 may be coupled to the joint replacement implant 1050 via an attachment feature (not shown), such as a post that may include a Morse taper, a snap-fit feature, a tab feature, a dovetail feature, a sleeve-in-sleeve feature design, etc.

In some embodiments, the joint replacement implant 1050 may include a shaft 1005 having a proximal end, a distal end, a threaded shaft portion, and a helical thread 1010 disposed about the shaft 1005 defining a length of the threaded shaft portion.

In some embodiments, the shaft 1005 may also include one or more transverse passageways (not shown) formed therein and configured to receive one or more or pins or fasteners (not shown) therethrough to provide additional stabilization for the joint replacement implant 1050. It will be understood that the one or more pins or fasteners may (or may not) include any thread configuration, feature, or morphology described or contemplated herein with respect to any fastener, bone implant, or joint replacement implant to achieve optimal fixation within a given bone, tissue, bone cement, bone augment material, etc. Moreover, it will also be understood that the one or more pins fasteners may be utilized in conjunction with (or within) any system, method, procedure, or instrumentation described or contemplated herein.

In some embodiments, the distal end of the shaft 1005 may be rounded or may comprise a hemi-spherical shape.

In some embodiments, the shaft 1005 and/or the helical thread 1010 may also include one or more self-tapping features or cutting flutes (not shown).

In some embodiments, the helical thread 1010 may include a concave undercut surface angled towards one of the proximal end and the distal end of the shaft 1005. However, it will also be understood that the joint replacement implant 1050 may (or may not) include any thread configuration, feature, or morphology described or contemplated herein with respect to any fastener, bone implant, or joint replacement implant to achieve optimal fixation within a given bone, tissue, bone cement, bone augment material, etc. For example, in some embodiments the helical thread 1010 may comprise standard or inverted threading, a “dual start” thread configuration, tapered helical threading, crescent shapes, etc. Moreover, it will also be understood that the joint replacement implant 1050 may be utilized in conjunction with (or within) any system, method, procedure, or instrumentation described or contemplated herein.

In some embodiments, the joint replacement implant system 1000 may be utilized with bone cement, any type of bone augment material(s), any feature(s) that may facilitate bony ingrowth, etc., to provide additional stabilization for the joint replacement implant 1050.

In some embodiments, the articular member 1060 may comprise a distal femoral prosthesis configured to replace a distal portion of a femur after it has been prepared/resected.

In some embodiments, the articular member 1060 may comprise one or more artificial articular surfaces or articular surfaces 1065.

In some embodiments, the one or more articular surfaces may comprise one or more convex (or concave) articular surfaces.

In some embodiments, the one or more convex articular surfaces of the articular member 1060 may be shaped to be received within one or more concave articular surfaces formed in the tibial insert prosthesis shown in FIGS. 9E-9H (or vice versa).

In some embodiments, the articular member 1060 may be removably couplable with the joint replacement implant 1050.

In some embodiments, the articular member 1060 may be integrally formed with the joint replacement implant 1050.

In some embodiments, the joint replacement implant 1050 may comprise a modular stem system comprising one or more intermediary components or one or more distal femur intermediary components (not shown), and the articular member 1060 may be couplable with at least one of the one or more intermediary components to dispose the articular member 1060 at the proximal end of the joint replacement implant 1050.

In some embodiments, the one or more intermediary components may include one of: a Morse taper feature, a snap-fit feature, a tab feature, a dovetail feature, a sleeve-in-sleeve feature, etc., intermediate the joint replacement implant 1050 and the articular member 1060.

In some embodiments, the one or more intermediary components may (or may not) include any thread configuration, feature, or morphology described or contemplated herein with respect to any fastener, bone implant, or joint replacement implant to facilitate coupling the articular member 1060 to the joint replacement implant 1050.

In some embodiments, a method for implanting the joint replacement implant system 1000 shown in FIG. 10 may include: resecting a portion of distal femur and/or forming a bone tunnel into the distal femur with a saw tool, a bone remover tool, a reamer tool, a broach tool, a drill tool, etc., (not shown); tapping a helical bone thread about the bone tunnel with a tapping tool (not shown), or utilizing self-tapping features on the joint replacement implant 950 to form the helical bone thread about the bone tunnel during insertion; inserting the joint replacement implant 1050 into the prepared bone tunnel; and coupling the articular member 1060 to the proximal end of the joint replacement implant 1050.

FIGS. 11A-11C illustrate a joint replacement implant system 1100 for a distal tibia 1180, according to embodiments of the present disclosure.

In some embodiments, the joint replacement implant system 1100 may include a joint replacement implant 1150, which may be coupled to an articular member 1160 via an attachment feature 1190 disposed at a proximal end of the joint replacement implant 1150.

In some embodiments, the attachment feature 1190 may comprise a distal tibial tray, tray feature, or intermediary tray component configured to couple the articular member 1160 to the joint replacement implant 1150.

In some embodiments, the articular member 1060 may be removably couplable with the attachment feature 1190 and/or the joint replacement implant 1150.

In some embodiments, the articular member 1160 may comprise an ankle insert prosthesis.

In some embodiments, the articular surface 1165 may comprise one or more concave (or convex) artificial articular surfaces formed in the ankle insert prosthesis or one or more convex artificial articular surfaces formed on the ankle insert prosthesis.

In some embodiments, the articular member 1160 may be integrally formed with the attachment feature 1190 and/or the joint replacement implant 1150.

In some embodiments, the joint replacement implant 1150 may include a shaft 1105 having a proximal end, a distal end, a threaded shaft portion, and a helical thread 1110 disposed about the shaft 1105 defining a length of the threaded shaft portion.

In some embodiments, the shaft 1105 may also include one or more transverse passageways formed therein and configured to receive one or more or pins or fasteners 1140 therethrough to provide additional stabilization for the joint replacement implant 1150, as shown in FIGS. 11B and 11C. It will be understood that the one or more pins or fasteners 1140 may (or may not) include any thread configuration, feature, or morphology described or contemplated herein with respect to any fastener, bone implant, or joint replacement implant to achieve optimal fixation within a given bone, tissue, bone cement, bone augment material, etc. Moreover, it will also be understood that the one or more pins fasteners may be utilized in conjunction with (or within) any system, method, procedure, or instrumentation described or contemplated herein.

In some embodiments, the distal end of the shaft 1105 may be rounded or may comprise a hemi-spherical shape.

In some embodiments, the shaft 1105 and/or the helical thread 1110 may also include one or more self-tapping features or cutting flutes (not shown).

In some embodiments, the helical thread 1110 may include a concave undercut surface angled towards one of the proximal end and the distal end of the shaft 1105. However, it will also be understood that the joint replacement implant 1150 may (or may not) include any thread configuration, feature, or morphology described or contemplated herein with respect to any fastener, bone implant, or joint replacement implant to achieve optimal fixation within a given bone, tissue, bone cement, bone augment material, etc. For example, in some embodiments the helical thread 1110 may comprise standard or inverted threading, a “dual start” thread configuration, tapered helical threading, crescent shapes, etc. Moreover, it will also be understood that the joint replacement implant 1150 may be utilized in conjunction with (or within) any system, method, procedure, or instrumentation described or contemplated herein.

In some embodiments, the joint replacement implant system 1100 may be utilized with bone cement, any type of bone augment material(s), any feature(s) that may facilitate bony ingrowth, etc., to provide additional stabilization for the joint replacement implant 1150.

In some embodiments, the articular member 1160 may comprise a distal tibia prosthesis configured to replace a distal portion of a tibia after it has been prepared/resected.

In some embodiments, the articular member 1160 may comprise an articular surface 1165.

In some embodiments, the articular surface may comprise a concave articular surface.

In some embodiments, the articular surface may comprise a convex articular surface.

In some embodiments, the articular member 1160 may be integrally formed with the joint replacement implant 1150.

In some embodiments, the articular member 1160 may be removably couplable with the joint replacement implant 1150 and/or the attachment feature 1190.

In some embodiments, the joint replacement implant 1150 may comprise a modular stem system comprising one or more intermediary components or one or more distal tibia intermediary components (not shown), and the articular member 1160 may be couplable with at least one of the one or more intermediary components to dispose the articular member 1160 at the proximal end of the joint replacement implant 1150.

In some embodiments, the one or more intermediary components may include one of: a Morse taper feature, a snap-fit feature, a tab feature, a dovetail feature, a sleeve-in-sleeve feature, etc., intermediate the joint replacement implant 1150 and the articular member 1160.

In some embodiments, the one or more intermediary components may include the attachment feature 1190, distal tibial tray, tray feature, or intermediary tray component configured to couple the articular member 1160 to the joint replacement implant 1150.

In some embodiments, the one or more intermediary components may (or may not) include any thread configuration, feature, or morphology described or contemplated herein with respect to any fastener, bone implant, or joint replacement implant to facilitate coupling the articular member 1160 to the joint replacement implant 1150.

In some embodiments, a method for implanting the joint replacement implant system 1100 shown in FIGS. 11A-11C may include: resecting a portion of distal tibia and/or forming a bone tunnel into the distal tibia with a saw tool, a bone remover tool, a reamer tool, a broach tool, a drill tool, etc., (not shown); tapping a helical bone thread about the bone tunnel with a tapping tool (not shown), or utilizing self-tapping features on the joint replacement implant 1150 to form the helical bone thread about the bone tunnel during insertion; inserting the joint replacement implant 1150 into the prepared bone tunnel; and coupling the articular member 1160 to the proximal end of the joint replacement implant 1150.

Any procedures or methods disclosed herein comprise one or more steps or actions for performing the described procedure or method. The method steps and/or actions may be interchanged with one another. In other words, unless a specific order of steps or actions is required for proper operation of the embodiment, the order and/or use of specific steps and/or actions may be modified. Moreover, any of the methods or procedures described herein may be further modified by omitting, deleting, and/or adding any of the method, procedure steps, or actions described or contemplated herein.

Any of the fasteners, bone implants, joint arthroplasty implants, articular members, etc., described or contemplated herein may be configured for removal and replacement during a revision procedure by simply unscrewing and removing the bone implant from the bone in which the bone implant resides. Moreover, the bone implants described herein may advantageously be removed from bone without removing any appreciable amount of the bone during the removal process in order to preserve the bone. In this manner, bone implants may be mechanically integrated with the bone, while not being permanently affixed to the bone, or integrated via bony ingrowth, in order to provide an instant and removable connection between the bone implant and the bone. Accordingly, revision procedures utilizing the bone implants described herein can result in less trauma to the bone with improved patient outcomes. However, it will also be understood that any of the bone implants described or contemplated herein may also be permanently or semi-permanently affixed to a bone via a bone cement (or any type of bone augment material), bony ingrowth, etc., as desired.

It will be understood than any feature or group of features described or contemplated herein with respect to any implant, system, method, or instrument may be combined in any fashion with any other implant, system, method, or instrument that is described or contemplated herein in order to make any number of different implant, system, method, or instrument configurations.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, drawing, or description thereof for the purpose of streamlining the present disclosure. This method of disclosure, however, is not to be interpreted as reflecting an intention that any embodiment or claim require more features than those expressly recited in that embodiment or claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112. It will be apparent to those having skill in the art that changes may be made to the details of the above-described embodiments without departing from the underlying principles set forth herein.

The phrases “connected to”, “coupled to”, and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure or the appended claims are not limited to the precise configuration and components disclosed herein. Various modifications, changes, and variations which will be apparent to those skilled in the art may be made in the arrangement, operation, and details of the devices, instruments, systems, and methods disclosed herein.

1. A joint replacement implant comprising:
a shaft comprising:
a proximal end;
a distal end; and
a threaded shaft portion;
a helical thread disposed about the shaft defining a length of the threaded shaft portion, wherein the helical thread comprises a concave undercut surface angled towards one of the proximal end and the distal end of the shaft; and
an attachment feature at the proximal end of the joint replacement implant,
wherein:
the attachment feature is configured to couple an articular member to the joint replacement implant;
the articular member comprises at least one artificial articular surface; and
the at least one artificial articular surface is shaped to replace at least a portion of a natural articular surface of a joint of a patient.

2. The joint replacement implant of claim 1, wherein the attachment feature comprises at least one of:
a post;
a Morse taper feature;
a passageway;
a dovetail feature;
a tray feature;
a snap-fit feature; and
a tab feature.

3. The joint replacement implant of claim 1, further comprising an intermediary component couplably disposed intermediate the attachment feature and the articular member.

4. The joint replacement implant of claim 3, wherein the intermediary component comprises at least one of:
an intermediary neck component; and
an intermediary tray component.

5. The joint replacement implant of claim 1 configured to receive at least one of:
a pin;
a fastener; and
a keel,
wherein the pin, the fastener, and the keel are configured to stabilize the joint replacement implant relative to a bone of the patient.

6. The joint replacement implant of claim 1, wherein the shaft of the joint replacement implant is shaped to be at least partially received within an intramedullary canal of a bone of the patient.

7. The joint replacement implant of claim 1, wherein:
the shaft comprises a tapered shaft portion; and
the helical thread comprises a tapered helical thread disposed about the tapered shaft portion.

8. A joint replacement implant system comprising:
a joint replacement implant comprising:
a shaft comprising:
a proximal end;
a distal end; and
a threaded shaft portion; and
a helical thread disposed about the shaft defining a length of the threaded shaft portion, wherein the helical thread comprises a concave undercut surface angled towards one of the proximal end and the distal end of the shaft; and
an articular member coupled to the proximal end of the joint replacement implant;
wherein:
the articular member comprises at least one artificial articular surface; and
the at least one artificial articular surface is shaped to replace at least a portion of a natural articular surface of a joint of a patient.

9. The joint replacement implant system of claim 8, further comprising an attachment feature at the proximal end of the joint replacement implant configured to couple the articular member to the joint replacement implant.

10. The joint replacement implant system of claim 9, further comprising an intermediary component disposed intermediate the attachment feature and the articular member and configured to couple therebetween.

11. The joint replacement implant system of claim 8, further comprising at least one of:
a pin;
a fastener; and
a keel,
wherein the pin, the fastener, and the keel are configured to stabilize the joint replacement implant relative to a bone of the patient.

12. The joint replacement implant system of claim 8, wherein:
the articular member comprises a femoral head prosthesis; and
the artificial articular surface comprises a convex semi-spherical artificial articular surface.

13. The joint replacement implant system of claim 8, wherein:
the articular member comprises a tibial insert prosthesis; and
the artificial articular surface comprises one or more concave artificial articular surfaces.

14. The joint replacement implant system of claim 8, wherein:
the articular member comprises an ankle insert prosthesis; and
the artificial articular surface comprises at least one of a concave artificial articular surface and a convex artificial articular surface.

15. A method for replacing at least a portion of a natural articular surface of a joint of a patient comprising:
preparing a bone of the patient to receive a joint replacement implant therein; and
inserting the joint replacement implant into the bone of the patient;
wherein:
the joint replacement implant comprises:
a shaft comprising:
a proximal end;
a distal end; and
a threaded shaft portion;
a helical thread disposed about the shaft defining a length of the threaded shaft portion, wherein the helical thread comprises a concave undercut surface angled towards one of the proximal end and the distal end of the shaft; and
an attachment feature at the proximal end of the joint replacement implant;
wherein:
the attachment feature is configured to couple an articular member to the joint replacement implant;
the articular member comprises at least one artificial articular surface; and
the at least one artificial articular surface is shaped to replace at least a portion of the natural articular surface of the joint of the patient.

16. The method of claim 15, wherein preparing the bone to receive the joint replacement implant comprises:
forming a bone tunnel in the bone configured to receive at least a portion of the joint replacement implant therein.

17. The method of claim 16, further comprising:
tapping a helical bone thread about the bone tunnel formed in the bone.

18. The method of claim 17, wherein inserting the joint replacement implant into the bone further comprises:
placing the helical thread of the joint replacement implant adjacent the helical bone thread tapped about the bone tunnel; and
rotating the joint replacement implant relative to the bone tunnel to:
insert the helical thread into the helical bone thread; and
insert the joint replacement implant into the bone tunnel.

19. The method of claim 16, wherein inserting the joint replacement implant into the bone further comprises:
placing the helical thread of the joint replacement implant adjacent the bone tunnel; and
rotating the joint replacement implant relative to the bone tunnel to:
form a helical bone thread about the bone tunnel via one or more self-tapping features of the joint replacement implant; and
insert the joint replacement implant into the bone tunnel.

20. The method of claim 15, further comprising:
coupling the articular member to the attachment feature of the joint replacement implant.
